(19) United States
(12) Patent Application Publication
(10) Pub. No.: US 2024/0324932 A1
(43) Pub. Date: Oct. 3, 2024

(54) SYSTEM FOR SENSING EEG SIGNALS ON A LIVING BEING, AND SYSTEM FOR ELECTRICALLY STIMULATING TISSUE OF A LIVING BEING

(71) Applicant: Precisis GmbH, Heidelberg (DE)

(72) Inventor: Lasse JAGSCHIES, Heidelberg (DE)

(21) Appl. No.: 18/037,117

(22) PCT Filed: Nov. 24, 2021
(86) PCT No.: PCT/EP2021/082817

§ 371 Date: May 16, 2023

(30) Foreign Application Priority Data
Nov. 27, 2020 (DE) 10 2020 131 503.1

(51) Int. Cl. A61B 5/293; A61B 5/372; A61N 1/05; A61N 1/36

(52) U.S. Cl. CPC A61B 5/293; A61B 5/372; A61N 1/0529; A61N 1/36135

(57) ABSTRACT

The invention relates to a system for sensing EEG signals on a living being, wherein the system includes multiple EEG electrodes for picking up electrical signals on the living being and at least one evaluation device connected to the EEG electrodes, wherein the evaluation device is configured to carry out at least the following steps:
a) picking up EEG signals from the EEG electrodes,
b) calculating at least bipolar and quadrupolar field components of the electrical field in the living being, which is the cause of the EEG signals, from the EEG signals,
c) determining at least one physiological key value of the living being from the calculated at least bipolar and quadrupolar field components.

The invention relates to a system for sensing EEG signals on a living being, wherein the system includes multiple EEG electrodes for picking up electrical signals on the living being and at least one evaluation device connected to the EEG electrodes. Electroencephalography (EEG) is a method of medical diagnostics and neurological research for measuring the summed electrical activity of the brain by recording the voltage variations on the head surface. The diagnostic possibilities of known EEG sensing systems are limited. The diagnostic possibilities of patients are to be further improved by the present invention.

The invention additionally relates to a system for electrically stimulating tissue of a living being using electrical stimulation signals to generate desired physiological key values of the living being, in particular for the electrical stimulation of brain tissue of the living being, wherein the system includes multiple stimulation electrodes and at least one stimulation device connected to the stimulation electrodes. Using such a system, for example, the treatment of neurological diseases by means of electrostimulation can be carried out, as described, for example, in EP 2 038 004 B1. The treatment possibilities are to be further improved by the present invention.

This is achieved in a system for sensing EEG signals as described at the outset in that the evaluation device is configured to carry out at least the following steps:
a) picking up EEG signals from the EEG electrodes,
b) calculating at least bipolar and quadrupolar field components of the electrical field in the living being, which is the cause of the EEG signals, from the EEG signals,
c) determining at least one physiological key value of the living being from the calculated at least bipolar and quadrupolar field components.

Fully novel diagnostic possibilities on the basis of the EEG signals can be enabled by the targeted determination at least of bipolar and quadrupolar field components, possibly also of monopolar or higher-polar field components, e.g., a seizure detection (epileptic seizure) of the patient using a random forest algorithm. It is thus possible to automatically distinguish on the basis of the field components, for example, between the states “normal EEG”, “movement artifact”, “epileptic seizure”. The higher-polar field components can be determined by computer here on the basis of a multipolar expansion (series expansion, for example Taylor series).

The determined physiological key value of the living being can be displayed, for example, on a display device, for example a display screen. In this way, the system can be used as a diagnostic device for the physician.

The evaluation device for executing the mentioned steps can be a computer-controlled device, i.e., having a computer which executes a computer program. The mentioned steps are then carried out by the execution of the computer program.

According to one advantageous refinement of the invention, it is provided that the evaluation device is configured to calculate at least the bipolar and quadrupolar field components by superposition, for example by linear combination or also by nonlinear superposition, of respective bipolar EEG signals of respective pairs of EEG electrodes. This has the advantage that without changing the position of the EEG electrodes, the signals can be differently sensed and evaluated on the living being, solely by corresponding software and algorithms. In this way, the diagnostic possibilities are further improved, in particular with essentially unchanged mechanical arrangement of the EEG electrodes. The invention also comprises the possibility that the EEG electrodes are placed in a geometrically fixed arrangement in relation to one another, both externally on the head surface and also subgaleally, i.e., between the skull and the scalp of the living being. In the last-mentioned case, the possibilities for a change of the electrode position are greatly restricted in any case, so that expanded diagnostic possibilities are also created by the invention with such a subgaleal arrangement of the electrodes.

According to one advantageous refinement of the invention, it is provided that the evaluation device is configured to determine the direction of field components of the electrical field in the living being, which is the cause of the EEG signals, from the at least bipolar and quadrupolar field components in consideration of data about the geometric arrangement of the EEG electrodes on the living being. Expanded diagnostic possibilities can be provided by such a direction determination of the electrical field components. Moreover, a foundation for regulated neurostimulation is also created in this way, in which a desired field direction is to be generated by the stimulation signals. The direction of the field components determined by sensing the EEG signals can thus be used as a feedback signal for regulated neurostimulation.

According to one advantageous refinement of the invention, it is provided that the evaluation device is configured, on the basis of the determined physiological key value, to detect one of multiple physiological states of the living being and/or to generate a trigger signal for a stimulation system, a warning system, and/or a data recording system. It is thus possible to distinguish, for example, between the physiological states sleeping, awake, and pathophysiological states such as pre-ictal activity or ictal activity, neuron activity which permits a pain process to be concluded. Moreover, movement artifacts, muscle artifacts, in particular of the chewing muscle or eye muscle or ECG signals, can be differentiated from real neural signals. This can be used, for example, as a trigger signal for a stimulation system, in order to automatically activate a therapeutic neurostimulation, for example, in case of a seizure or activity which is accompanied by an increased probability of seizure or a pain process. The system can also, for example, be used as a warning system in a hospital or a care facility or at home or underway. An optical and/or acoustic signal element or a domestic emergency call system or a call via a mobile telephone can be triggered by the trigger signal, for example, using which a physician or a carer is sent for.

According to one advantageous refinement of the invention, it is provided that the evaluation device is configured to calculate further field components of the electrical field in the living being, which is the cause of the EEG signals, from the EEG signals, in particular monopolar and/or octopolar and/or higher-polar field components. The diagnostic possibilities can be further improved in this way. In particular, it is possible to distinguish the living being even more finely according to different physiological states.

A system for electrical stimulation, as described at the outset, can be further improved in that the stimulation device is configured to carry out at least the following steps:
a) reading in specification parameters of an electrical field to be generated by the stimulation signals in the tissue of the living being,
b) determining stimulation signals by which alternately at least one bipolar formed electrical field, a quadrupolar formed electrical field, or a superposition of bipolar and quadrupolar formed electrical field is generated in the tissue of the living being as a function of the specification parameters,
c) outputting the stimulation signals via the stimulation electrodes to the tissue of the living being.

In step b), current or voltage values of the stimulation signals to be output via a respective stimulation electrode can be computed for each individual stimulation electrode from the field components. The superposition of bipolar and quadrupolar field components can be carried out, for example, by linear combination of these field components or also by nonlinear superposition.

In this way, the treatment possibilities of patients by neurostimulation can be improved, since certain field forms or field directions of the electrical field can be generated deliberately according to the specification parameters. This permits a patient-specific treatment which can possibly also be adjusted with little effort in the course of the treatment, since no change of the position of the stimulation electrodes is required for this purpose. The adjustment can be carried out by changing the software or the specification parameters. The specification parameters can be transmitted, for example, in the case of a stimulation system implanted in the patient via a wireless interface into the stimulation system. The specification parameters can be determined by the physician after a corresponding diagnosis and/or can be determined entirely or partially automatically, for example from the results of preceding EEG examinations.

The stimulation device can be a computer-controlled device for executing the mentioned steps, i.e., having a computer which executes a computer program. The mentioned steps are then carried out by the execution of the computer program.

According to one advantageous refinement of the invention, it is provided that the stimulation device is configured to carry out at least the following steps:
a) calculating at least bipolar and quadrupolar field components of the electrical field to be generated in the tissue of the living being from the specification parameters,
b) determining current or voltage values of the stimulation signals to be output via a respective stimulation electrode for each individual stimulation electrode at least from the bipolar and quadrupolar field components.

This permits a very precise determination of the current or voltage values of the stimulation signals using computing steps that can be carried out rapidly by the computer.

According to an advantageous refinement of the invention, it is provided that the stimulation device is configured to emulate a direction, defined on the basis of the specification parameters, of field components of the electrical field to be generated by the stimulation signals in the tissue of the living being by superposition of at least bipolar and quadrupolar field components and a determination of current or voltage values of the stimulation signals to be output via a respective stimulation electrode for each individual stimulation electrode at least from the superposition of at least bipolar and quadrupolar field components in the tissue of the living being. The determination of the current or voltage values can be further improved in this way.

According to an advantageous refinement of the invention, it is provided that the stimulation device is configured to chronologically vary the spatial direction of the electrical field generated by the stimulation signals in the tissue of the living being by means of the superposition of at least bipolar and quadrupolar field components. The chronological variation can be carried out relatively slowly, for example over multiple treatment phases, or relatively quickly, for example multiple times during one treatment phase. Improved treatment possibilities by neurostimulation are created in this way.

According to one advantageous refinement of the invention, it is provided that the stimulation device is configured to generate further field components of the electrical field to be generated in the tissue of the living being from the specification parameters, in particular monopolar and/or octopolar and/or higher-polar field components. The treatment possibilities can be improved still further in this way.

According to one advantageous refinement of the invention, it is provided that the system has a graphic user interface, via which the user can define the electrical field to be generated by the stimulation signals in the tissue of the living being by graphic inputs, wherein the user interface is configured to determine from the graphic inputs the specification parameters to be read in by the stimulation device of the electrical field to be generated by the stimulation signals in the tissue of the living being. In this way, an interface which is simple to operate for the input of the specification parameters is provided to the user, for example the physician. For example, the desired electrical field to be generated can be defined by graphic inputs, for example by characters of a desired field. Thus, for example, the location, the direction, and the intensity of the electrical field to be generated and the change of these parameters over time can be input via the user interface.

According to an advantageous refinement of the invention, it is provided that the system has a subsystem for sensing EEG signals on the living being, wherein the subsystem includes multiple EEG electrodes for picking up electrical signals on the living being and at least one evaluation device connected to the EEG electrodes, wherein the evaluation device is configured to carry out at least the following steps:
a) picking up EEG signals from the EEG electrodes,
b) determining at least one physiological key value of the living being from the EEG signals.

This has the advantage that the treatment by neurostimulation can be monitored directly on the basis of the EEG signals. In this way, for example, the treatment success can be assessed immediately and an adjustment can be carried out quickly in the case of undesired effects. The subsystem can be designed, for example, as a system of the type described at the outset for sensing EEG signals. The stimulation device can be formed as a common unit with the evaluation device.

According to one advantageous refinement of the invention, it is provided that the system is configured, in the event of a deviation between the determined and desired physiological key values of the living being, to automatically adjust the determination of the stimulation signals in such a way that the deviation between the determined and desired physiological key values is decreased or eliminated. In this way, the above-mentioned regulated neurostimulation can take place, i.e., the physiological key values determined by the EEG signals have a direct effect in terms of a feedback signal on the output stimulation signals, which are automatically adjusted so that as a result the desired physiological key values are generated by the stimulation signals. In this way, a self-adaptive stimulation system can be provided.

According to one advantageous refinement of the invention, it is provided that the evaluation device is configured to carry out at least the following steps:
a) calculating at least bipolar and quadrupolar field components of the electrical field in the living being, which is the cause of the EEG signals, from the EEG signals as a physiological key value of the living being,
b) providing the calculated at least bipolar and quadrupolar field components to a comparison device,
c) wherein the comparison device is configured to compare the calculated at least bipolar and quadrupolar field components to the at least bipolar and quadrupolar field components desired on the basis of the specification parameters and to signal a deviation between the calculated and desired field components.

In this way, the self-adaptive neurostimulation can be further improved and made even more precise. The system can be configured, in the event of a deviation between the calculated and desired field components signaled by the comparison device, to automatically adjust the determination of the stimulation signals in such a way that the deviation between the calculated and desired field components is reduced or eliminated.

It is possible that separate EEG electrodes and stimulation electrodes are used in each case. According to one advantageous refinement of the invention, it is provided that some or all EEG electrodes are formed by some or all stimulation electrodes. In this way, the overall system is simplified and in particular the strain on the patient is minimized. The electrodes used can be used alternately, for example, in the time multiplexing method as an EEG electrode and as a stimulation electrode.

According to one advantageous refinement of the invention, it is provided that the EEG electrodes and/or the stimulation electrodes are part of an electrode unit on which the EEG electrodes and/or the stimulation electrodes are fastened in a fixed geometrical arrangement in relation to one another. This has the advantage that the mechanical parameters of the structure of the electrode unit are invariable and are known to the system. In this way, various calculation steps are simplified, for example for the calculation of the direction of the electrical field. Moreover, the fastening or implantation of the electrode unit on the patient is also simplified in this way, in particular with subgaleal attachment.

According to one advantageous refinement of the invention, it is provided that the electrode unit includes at least
a) one central electrode and multiple surrounding electrodes arranged around the central electrode, in particular having four surrounding electrodes in a pseudo-Laplace arrangement and/or
b) a 3-electrode or 4-electrode arrangement in series and/or
c) a large number of electrodes in a matrix arrangement.

This has the advantage that various types of electrical fields can be generated on the patient by the same electrode unit, in particular also fields going into the depth of the tissue.

According to one advantageous refinement of the invention, it is provided that the system includes an auxiliary electrode as a further EEG electrode and/or stimulation electrode, which is not fastened to the electrode unit or is at least arranged at a distance from the surrounding electrodes, which is at least five times the mean distance of the surrounding electrodes from the central electrode. The auxiliary electrode can be arranged, for example, on a housing of the stimulation device. Using such an auxiliary electrode, for example, monopolar field components of the electrical field can be generated or, if the auxiliary electrode is used as an EEG electrode, can be sensed.

According to one advantageous refinement of the invention, it is provided that the system is configured so that the EEG electrodes and/or the stimulation electrodes are arranged between the skull and the scalp of the living being. In this way, on the one hand, particularly effective neurostimulation and thus the best possible treatment possibilities can be created. In comparison to electrodes implanted below the skullcap, the stress for the patient is significantly less, however.

Insofar as a computer is mentioned, this can be configured to execute a computer program, for example in the meaning of software. The computer can be designed as a commercially-available computer, e.g., as a PC, laptop, notebook, tablet, or smart phone, or as a microprocessor, microcontroller, or FPGA, or as a combination of such elements.

The invention will be explained in more detail hereinafter on the basis of exemplary embodiments using drawings.

In the figures:

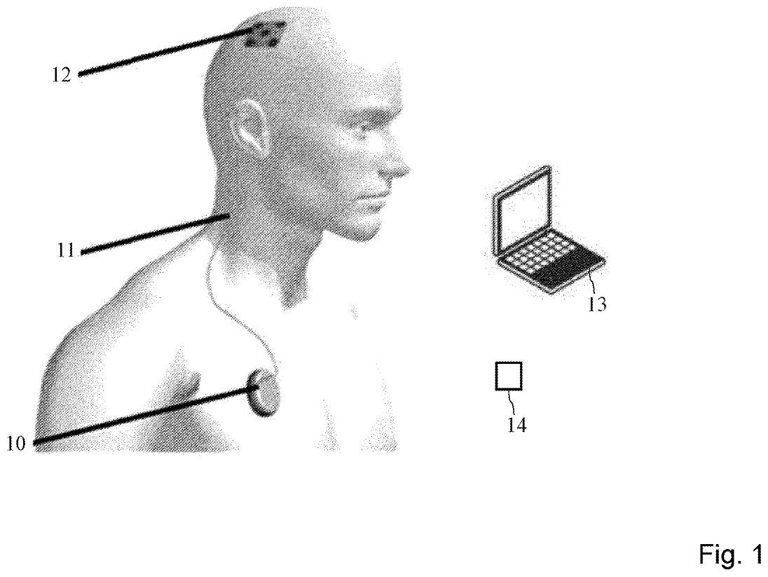

FIG. 1 shows a system according to the invention implanted on a human and

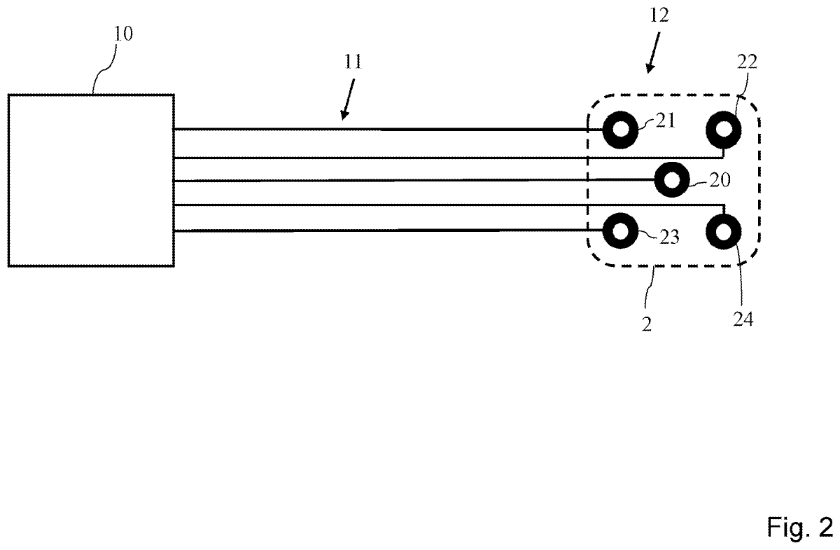

FIG. 2 shows the components of the system according to FIG. 1 and

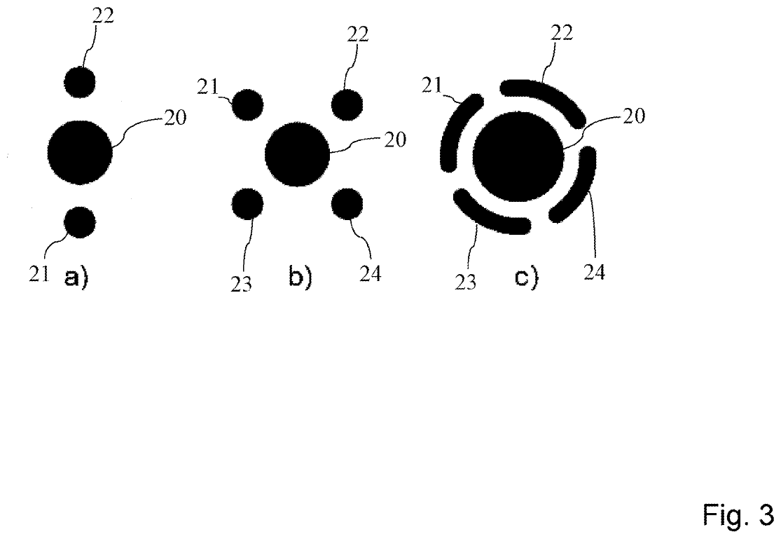

FIG. 3 shows examples of electrode units and

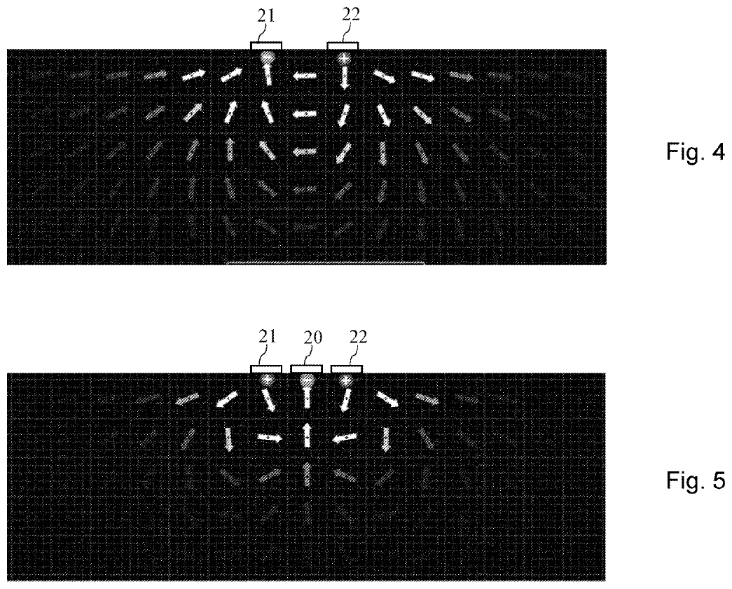

FIG. 4 shows a bipolar formed electrical field and

FIG. 5 shows a quadrupolar formed electrical field and

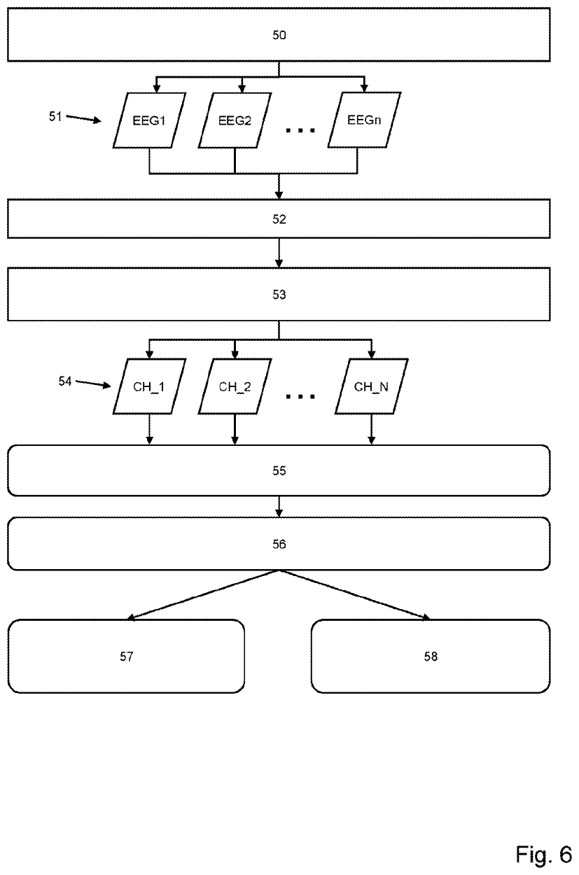

FIG. 6 shows a first embodiment of EEG evaluation steps and

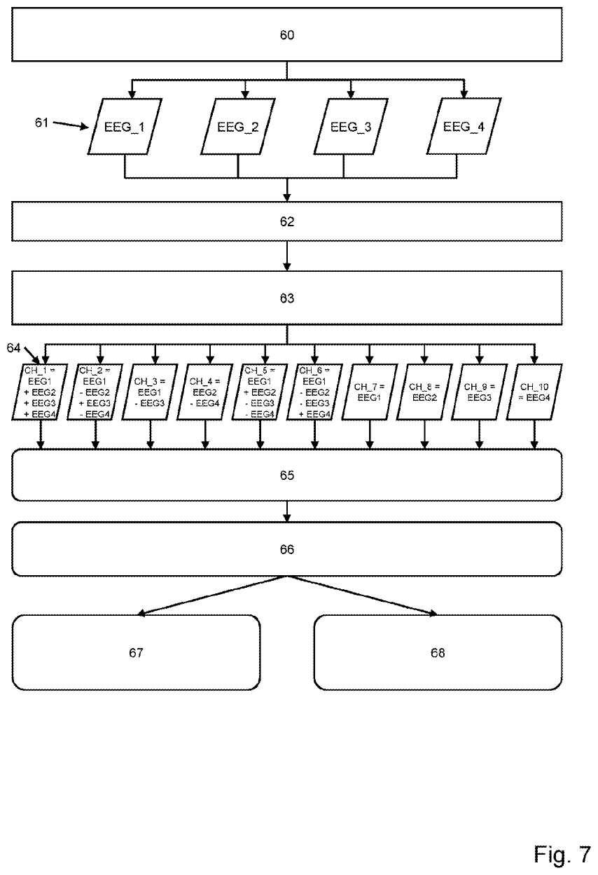

FIG. 7 shows a second embodiment of EEG evaluation steps and

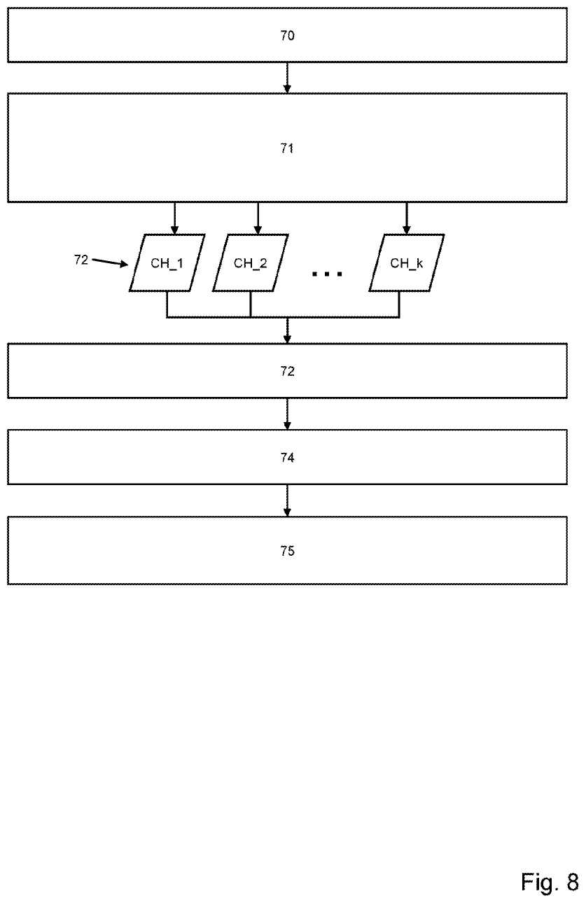

FIG. 8 shows a first embodiment of stimulation steps and

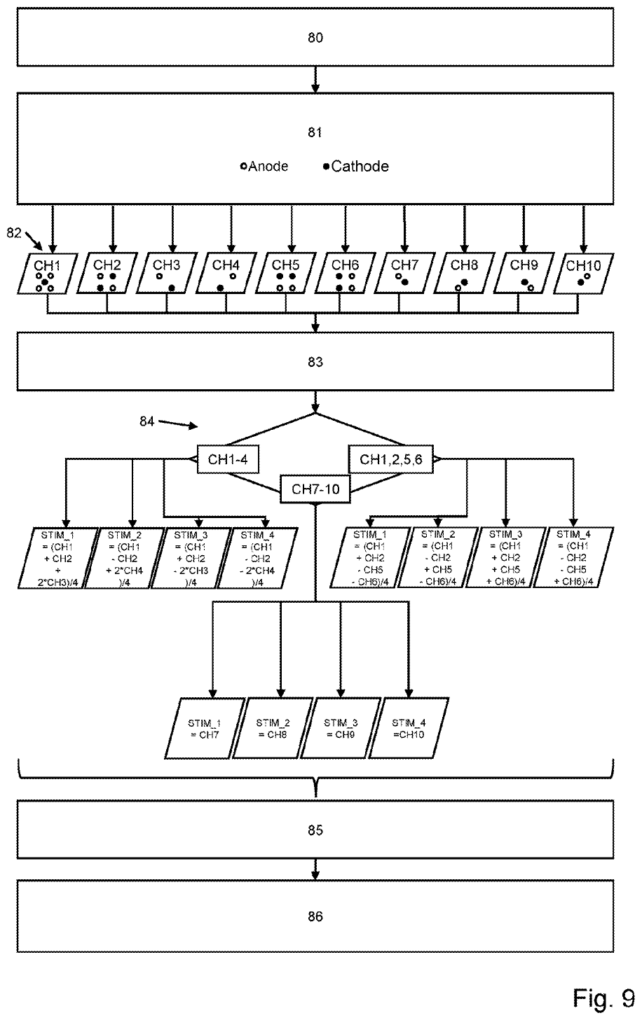

FIG. 9 shows a second embodiment of stimulation steps and

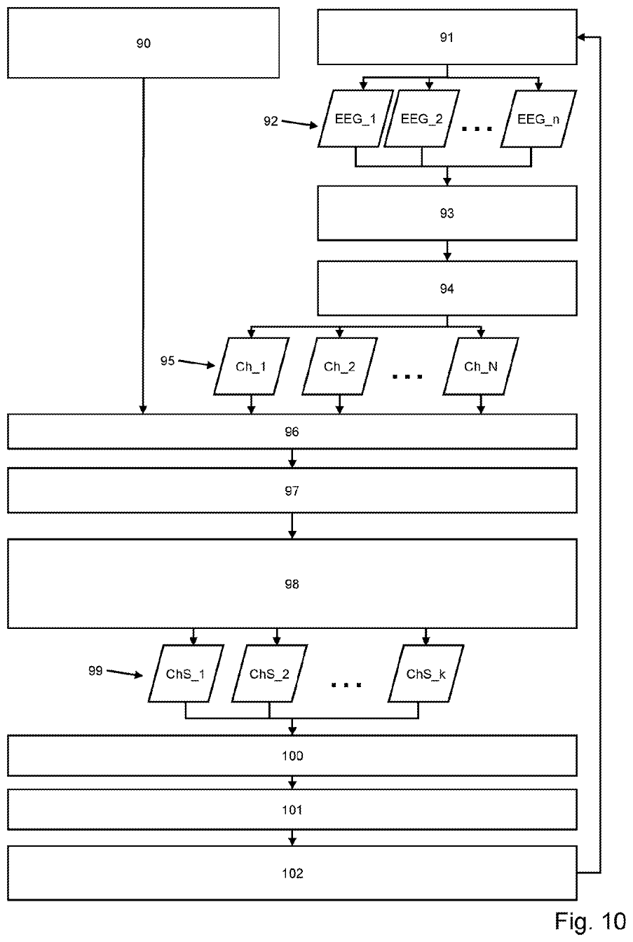

FIG. 10 shows steps of a stimulation controlled by recorded EEG signals.

The disclosed device is completely implantable and stimulates defined areas of the brain. It can be used to treat various neurological disorders, among others refractory epilepsy, wherein the device prophylactically prevents the occurrence of epileptic seizures in that it continuously emits stimulation pulses. Long-term stimulation enables changes in neural networks and in the plasticity, so that a “modulation effect” occurs. The brain is thus less susceptible to epileptic seizures and the patient having epilepsy can live a higher quality of life.

The system according to the invention, as shown in FIG. 1, includes three parts 10, 11, 12 completely implantable in the patient and can be assisted and/or controlled by non-implanted parts 13, 14. The implantable parts comprise a central unit 10, a connecting line 11, and an electrode unit 12.

The electrode unit 12 includes multiple electrodes which are mounted on a substrate that is implanted in the subgaleal area (below the scalp but outside the skull). Each of these individual electrodes can be individually controlled to generate a symmetrical electrical field under the electrodes, which guides the current perpendicular to the electrode surface and thus optimizes the penetration depth.

The central unit 10, which can be implanted, for example, below the clavicle, has an energy source, for example an accumulator or a battery, and a control electronics unit, which can also contain, for example, a circuit for the charge equalization at the electrodes.

The connecting line 11, which contains multiple cables, connects the central unit 10 to the electrode unit 12. The connecting line 11 is implanted under the skin.

A device 13 (not implanted) enables trained medical personnel to set the stimulation parameters according to the individual requirements of the patient and to test the functionality of the central unit 10 (battery service life, impedance) and to allow access to data which were recorded thereby.

A manual command device 14 (not implanted) enables the patient to record the event of a seizure, to check the battery status, to trigger the treatment using preset stimulation pulses, and to switch off the system in emergency.

FIG. 2 shows the electrode unit 12 having an advantageous pseudo-Laplace arrangement of the electrodes of the neurostimulation system. A substrate 2 comprises a central electrode 20 and four peripheral electrodes 21, 22, 23, 24. The central electrode 20 can be located between the peripheral electrodes 21, 22, 23, 24, for example in the center of the substrate 2. The peripheral electrodes 21, 22, 23, 24 thus surround the central electrode 20.

The system according to FIG. 1 can be designed as a system for sensing EEG signals, as a system for electrical stimulation of tissue of the living being using electrical stimulation signals, or as a combined device, which fulfills both functions. In the first-mentioned case, the electrodes of the electrode unit 12 are used to pick up the EEG signals. Accordingly, the central unit 10 has the function of the evaluation device. In the second-mentioned case, the electrodes are used as stimulation electrodes. In this case, the central unit 10 has the function of the stimulation device. In the combined device, the electrodes can be used both to pick up the EEG signals and to output the stimulation pulses. In this case, the central unit 10 has both the function of the evaluation device and of the stimulation device.

FIG. 3 shows further advantageous designs of the electrode unit 12. In example a), one central electrode 20 and two peripheral electrodes 21, 22 are provided. Examples b) and c) each show pseudo-Laplace arrangements having a central electrode and four peripheral electrodes. In contrast to the embodiment of FIG. 2, the peripheral electrodes 21, 22, 23, 24 each have smaller surfaces than the central electrode 20 here. In example b), all electrodes are made circular. In example c), only the central electrode 20 is circular, the peripheral electrodes have a shape like a circular arc, which is rounded in the end areas. In all examples a), b), c), the central electrode 20 is concentrically enclosed by the peripheral electrodes.

In FIG. 4, a bipolar formed electrical field generated by the electrode unit 12 in the tissue of the patient can be seen.

FIG. 5 shows a quadrupolar formed electrical field generated by the electrode unit 12 in the tissue of the patient.

An evaluation algorithm for sensing and evaluating EEG signals, which are picked up via the electrodes, is described in general form on the basis of FIG. 6. It is presumed that a number of n physical EEG channels EEG1 to EEGn are available, so that n bipolar EEG signals are picked up independently.

In a step 50, the EEG signals are picked up via the channels EEG1 to EEGn on the living being. In steps 51, 52, the recorded data of the channels EEG1 . . . EEGn are supplied to the evaluation device for processing. In a step 53, the data are processed in such a way that a number of N monopolar and/or bipolar and/or quadrupolar and/or octopolar and/or higher-polar field components of the electrical field in the living being, which is the cause of the EEG signals, is determined from the EEG signals in channels CH_1 to CH_N, for example as a linear combination or other superposition of the signals picked up in a bipolar manner on the living being of the channels EEG1 to EEGn. N can be equal or unequal to n, in particular N>n can apply.

The channels CH_1 to CH_N calculated in this way are transferred in a step 54 to a further evaluation step 55, in which one or more physiological key values of the living being, on which the EEG signals were picked up, are determined from the channels CH_1 to CH_N. In particular, a physiological key value can be determined which is to be monitored by the system. In a following step 56, the channels CH_1 to CH_N and/or the previously determined physiological key values are assessed according to defined categories or classified in such categories.

Hereafter, in an optional step 57, for example, a trigger signal for a stimulation system, a warning system, or a data recording system can be generated. In an optional step 58, the data from the channels CH_1 to CH_N and/or the determined key values can be transferred via an interface to a further system. In this way, the data can also be made available to other systems.

FIG. 7 shows a more specific example of the algorithm described on the basis of FIG. 6 for the case that a number of n=4 physical EEG channels EEG1 to EEG4 are available, so that 4 bipolar EEG signals are picked up independently. In a step 60, the EEG signals are picked up via the channels EEG1 to EEG4 on the living being. In steps 61, 62, the recorded data of the channels EEG1 . . . EEG4 are supplied to the evaluation device for processing. In a step 63, the data are processed in such a way that a number of N=10 monopolar and/or bipolar and/or quadrupolar and/or octopolar and/or higher-polar field components of the electrical field in the living being, which is the cause of the EEG signals, is determined from the EEG signals in channels CH_1 to CH_10. Step 64 shows by way of example the determination equations usable for this purpose.

In step 65, the channels CH_1 to CH_10 are evaluated to determine the at least one physiological key value of the living being from the calculated at least bipolar and quadrupolar field components, analogously to step 55. Step 66 corresponds to step 56 for the case of ten channels CH_1 to CH_10. Step 67 corresponds to step 57. Step 68 corresponds to step 58.

FIG. 8 shows a stimulation algorithm which can be executed in a stimulation device of a system for electrical stimulation of tissue of a living being using electrical stimulation signals, in general form for a number of k physical stimulation channels. In a step 70, input values are input, for example via a graphic user interface. For example, data for the location, the direction, the intensity of the electrical field to be generated by the electrical stimulation signals, and the change of these parameters over time are input via the user interface. Specification parameters for the stimulation device can then be determined by the input values.

In a step 71, for the k different physical stimulation channels CH_1 to CH_k, the individual parameters of the stimulation signals are determined, in particular the chronological sequence of voltage or current amplitudes, so that as a result in the living being an electrical field is generated in accordance with the specification parameters with respect to location, direction, intensity, and chronological development. The individual stimulation signals are determined in such a way that the superposition of the electrical fields generated thereby ultimately provides the desired shape and chronological development of the electrical field in the living being. For this purpose, for example, parameters for a bipolar formed electrical field, a quadrupolar formed electrical field, or a superposition of bipolar and quadrupolar formed electrical field are generated. In a step 72, the corresponding output channels are defined, i.e., the electrodes to which the stimulation signals are to be applied and their polarity, i.e., whether the electrode is used as an anode or cathode. In this case, for example, n output channels STIM_1 to STIM_n are defined, wherein n can be equal to k or n can be unequal to k, in particular with n>k. In a step 73, for each physical pair of anode and cathode of these output channels STIM_1 to STIM_n, a chronological sequence of current or voltage amplitudes is determined as a linear combination or another superposition from the parameters of the stimulation signals determined for the stimulation channels CH_1 to CH_k in step 71.

In a step 74, the chronological sequence of current and voltage amplitudes of the output channels STIM_1 to STIM_n is transferred from the computer to an output interface of the stimulation device. In a step 75, at the output interface, a control of the physical stimulation channels CH_1 to CH_k is carried out in accordance with the chronological sequence of the current or voltage amplitudes determined in step 73, possibly carried out according to a predetermined stimulation sequence or a trigger signal by an event detector, so that the electrical stimulation signals are ultimately admitted to the living being.

FIG. 9 shows a more specific embodiment of the general algorithm according to FIG. 8 on the basis of an example of k=4 physical stimulation channels STIM_1 to STIM_4, to which signals are applied to generate, for example, n=10 bipolar or quadrupolar output channels CH1 to CH10. In this case, for example, 10 bipolar or quadrupolar output channels CH1 to CH10 are defined, i.e., different electrodes of the electrode unit are each assigned to a specific output channel. Step 82 shows by way of example on the basis of the graphic symbols reproduced therein the assignment of the respective electrodes of an electrode unit having a central electrode and four surrounding electrodes in a pseudo-Laplace arrangement, as illustrated on the basis of FIG. 2.

First, the input values are again input in step 80, analogously to step 70 of FIG. 8. In step 81, analogously as explained in step 71, the corresponding parameters of the stimulation signals of the output channels CH1 to CH10 are determined, in particular the chronological sequence of voltage or current amplitudes with respect to location, direction, intensity, and chronological development, so that as a result an electrical field is generated in the living being in accordance with the specification parameters with respect to location, direction, intensity, and chronological development. A chronological sequence of the current or voltage amplitudes is determined in such a way that the superposition of the electrical fields, which are generated by the currents or voltages output via the 10 output channels, corresponds to the corresponding specification parameters.

In step 83, for each physical pair of anode and cathode of the electrode unit, the chronological sequence of the current or voltage amplitudes is determined from a linear combination of the channels CH1 to CH10. Steps 84 show the individual determination equations used for this purpose.

In a step 85, the chronological sequence of current or voltage amplitudes of the output channels CH1 to CH10 is transferred from the computer to an output interface of the stimulation device. In a step 86, a control of the physical stimulation channels STIM_1 to STIM_4 is carried out at the output interface in accordance with the chronological sequence of the current or voltage amplitudes determined in step 84, possibly carried out according to a predetermined stimulation sequence or a trigger signal from an event detector, so that the electrical stimulation signals are ultimately admitted to the living being.

FIG. 10 shows the steps of a stimulation controlled or regulated on the basis of recorded EEG signals, i.e., the stimulation signals are influenced on the basis of the fed back measurements via the EEG signals. In step 90, the specification parameters are input for the stimulation, analogously to steps 70 or 80. In step 94, N monopolar and/or bipolar and/or quadrupolar and/or octopolar and/or higher-polar channels CH_1 to CH_N are calculated using a linear combination of the neurological data EEG_1 to EEG_n. These neurological data EEG_1 to EEG_n are recorded by a subsystem in steps 91, 92, and 93. The sensing of the data EEG_1 to EEG_n can take place corresponding to FIG. 6, i.e., step 91 corresponds to step 50, step 92 corresponds to step 51, and step 93 corresponds to step 52. Step 94 corresponds to step 53. Step 95 corresponds to step 54.

In step 96, for each of channels CH_1 to CH_N, m features are calculated according to the m specification parameters, which are used to specify a desired or undesired neuron activity. In step 97, m comparison parameters PEQ_1j-PEQ_mj are calculated for each channel CH_1 to CH_N, which specify the difference between the present neuron activity and the desired or undesired neuron activity. In step 98, for k independent channels ChS_1 to ChS_k, a chronological sequence of stimulation current or stimulation voltage amplitudes is calculated. The following linear combination is used here:

$$\sum ij ( M\_ijk * PEQ\_ij * Ch\_j ) where i = 1 : m , j = 1 : N$$

M_k is a matrix of weighting factors for each channel ChS_k

The stimulation signals are then output in steps 99 to 102. Step 99 corresponds to step 72. Step 100 corresponds to step 73. Step 101 corresponds to step 74. Step 102 corresponds to step 75.

After step 102, the sequence continues again with step 91. The essential difference of the embodiment of FIG. 10 from FIG. 8 or 9 is thus the feedback of the measured EEG signals and their consideration in step 96 for the determination of the stimulation signals to be output.

1. A system for sensing EEG signals on a living being, wherein the system includes multiple EEG electrodes for picking up electrical signals on the living being and at least one evaluation device connected to the EEG electrodes, wherein the evaluation device is configured to carry out at least the following steps:
d) picking up EEG signals from the EEG electrodes,
e) calculating at least bipolar and quadrupolar field components of the electrical field in the living being, which is the cause of the EEG signals, from the EEG signals,
f) determining at least one physiological key value of the living being from the calculated at least bipolar and quadrupolar field components.

2. The system as claimed in claim 1, characterized in that the evaluation device is configured to calculate at least the bipolar and quadrupolar field components by superposition of respective bipolar EEG signals of respective pairs of EEG electrodes.

3. The system as claimed in any one of the preceding claims, characterized in that the evaluation device is configured to determine the direction of field components of the electrical field in the living being, which is the cause of the EEG signals, from the at least bipolar and quadrupolar field components in consideration of data about the geometric arrangement of the EEG electrodes on the living being.

4. The system as claimed in any one of the preceding claims, characterized in that the evaluation device is configured, on the basis of the determined physiological key value, to detect one of multiple physiological states of the living being and/or to generate a trigger signal for a stimulation system, a warning system, and/or a data recording system.

5. The system as claimed in any one of the preceding claims, characterized in that the evaluation device is configured to calculate further field components of the electrical field in the living being, which is the cause of the EEG signals, from the EEG signals, in particular monopolar and/or octopolar and/or higher-polar field components.

6. A system for the electrical stimulation of tissue of a living being using electrical stimulation signals, in particular for the electrical stimulation of brain tissue of the living being, wherein the system includes multiple stimulation electrodes and at least one stimulation device connected to the stimulation electrodes, wherein the stimulation device is configured to carry out at least the following steps:
d) reading in specification parameters of an electrical field to be generated by the stimulation signals in the tissue of the living being,
e) determining stimulation signals, by which alternately at least one bipolar formed electrical field, a quadrupolar formed electrical field, or a superposition of bipolar and quadrupolar formed electrical field is generated in the tissue of the living being, as a function of the specification parameters,
f) outputting the stimulation signals via the stimulation electrodes to the tissue of the living being.

7. The system as claimed in claim 6, characterized in that the stimulation device is configured to carry out at least the following steps:
c) calculating at least bipolar and quadrupolar field components of the electrical field to be generated in the tissue of the living being from the specification parameters,
d) determining current or voltage values of the stimulation signals to be output via a respective stimulation electrode for each individual stimulation electrode at least from the bipolar and quadrupolar field components.

8. The system as claimed in one of claims 6 to 7, characterized in that the stimulation device is configured to emulate a direction, defined on the basis of the specification parameters, of field components of the electrical field to be generated by the stimulation signals in the tissue of the living being by superposition of at least bipolar and quadrupolar field components and a determination of current or voltage values of the stimulation signals to be output via a respective stimulation electrode for each individual stimulation electrode at least from the superposition of at least bipolar and quadrupolar field components in the tissue of the living being.

9. The system as claimed in claim 8, characterized in that the stimulation device is configured to chronologically vary the spatial direction of the electrical field generated by the stimulation signals in the tissue of the living being by means of the superposition of at least bipolar and quadrupolar field components.

10. The system as claimed in any one of claims 6 to 9, characterized in that the stimulation device is configured to generate further field components of the electrical field to be generated in the tissue of the living being from the specification parameters, in particular monopolar and/or octopolar and/or higher-polar field components.

11. The system as claimed in any one of claims 6 to 10, characterized in that the system has a graphic user interface, via which the user can define the electrical field to be generated by the stimulation signals in the tissue of the living being by graphic inputs, wherein the user interface is configured to determine from the graphic inputs the specification parameters to be read in by the stimulation device of the electrical field to be generated by the stimulation signals in the tissue of the living being.

12. The system as claimed in any one of claims 6 to 11, characterized in that the system has a subsystem for sensing EEG signals on the living being, wherein the subsystem includes multiple EEG electrodes for recording electrical signals on the living being and at least one evaluation device connected to the EEG electrodes, wherein the evaluation device is configured to carry out at least the following steps:
c) picking up EEG signals from the EEG electrodes,
d) determining at least one physiological key value of the living being from the EEG signals.

13. The system as claimed in claim 12, characterized in that the system is configured, in the event of a deviation between the determined and desired physiological key values of the living being, to automatically adjust the determination of the stimulation signals in such a way that the deviation between the determined and desired physiological key values is reduced or eliminated.

14. The system as claimed in claim 12 or 13, characterized in that the evaluation device is configured to carry out at least the following steps:
d) calculating at least bipolar and quadrupolar field components of the electrical field in the living being, which is the cause of the EEG signals, from the EEG signals as a physiological key value of the living being,
e) providing the calculated at least bipolar and quadrupolar field components to a comparison device,
f) wherein the comparison device is configured to compare the calculated at least bipolar and quadrupolar field components to the at least bipolar and quadrupolar field components desired on the basis of the specification parameters and to signal a deviation between the calculated and desired field components.

15. The system as claimed in any one of claims 12 to 14, characterized in that the subsystem is designed as a system as claimed in any one of claims 1 to 5.

16. The system as claimed in any one of claims 12 to 15, characterized in that some or all EEG electrodes are formed by some or all stimulation electrodes.

17. The system as claimed in any one of the preceding claims, characterized in that the EEG electrodes and/or the stimulation electrodes are part of an electrode unit, on which the EEG electrodes and/or the stimulation electrodes are fastened in a fixed geometrical arrangement in relation to one another.

18. The system as claimed in claim 17, characterized in that the electrode unit includes at least
d) one central electrode and multiple surrounding electrodes arranged around the central electrode, in particular having four surrounding electrodes in a pseudo-Laplace arrangement and/or
e) a 3-electrode or 4-electrode arrangement in series and/or
f) a large number of electrodes in a matrix arrangement.

19. The system as claimed in claim 17 or 18, characterized in that the system includes an auxiliary electrode as a further EEG electrode and/or stimulation electrode, which is not fastened to the electrode unit or is at least arranged at a distance from the surrounding electrodes which is at least five times the mean distance of the surrounding electrodes from the central electrode.

20. The system as claimed in any one of the preceding claims, characterized in that the system is configured so that the EEG electrodes and/or the stimulation electrodes are arranged between the skull and the scalp of the living being.
